(19) United States
(12) Patent Application Publication
(10) Pub. No.: US 2022/0087798 A1
(43) Pub. Date: Mar. 24, 2022

(54) Oral Hygiene Assembly

(71) Applicant: Melanie Ritz, Hutchinson, MN (US)

(72) Inventor: Melanie Ritz, Hutchinson, MN (US)

(21) Appl. No.: 17/031,081

(22) Filed: Sep. 24, 2020

(51) Int. Cl. A61C 15/04; A61B 17/24

(52) U.S. Cl. CPC A61C 15/047; A61B 17/244

(57) ABSTRACT

An oral hygiene assembly for performing oral hygiene includes a handle that is elongated for reaching into a user's mouth. A vibration unit is integrated into the handle and the vibration unit has an output shaft extending outwardly from the handle. The output shaft vibrates when the vibration unit is turned on. A plurality of heads is provided and a respective one of the heads is removably attachable to the output shaft of the vibration unit such that the vibration vibrates the respective head.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to hygiene devices and more particularly pertains to a new hygiene device for performing oral hygiene.

### (2) Description of Related Art Including Information Disclosed Under 37 CFR 1.97 and 1.98

The prior art relates to hygiene devices including a powered dental cleaner that includes a string that coupled to a spool and which can be threaded through a flossing implement. The prior art discloses a flossing device that includes a flossing head which has a vibration unit integrated therein for vibrating the flossing head. The prior art discloses a vibrating oral hygiene device that includes a vibrating flossing head. The prior art discloses a powered toothbrush that includes a pair of rotating bristles.

### BRIEF SUMMARY OF THE INVENTION

An embodiment of the disclosure meets the needs presented above by generally comprising a handle that is elongated for reaching into a user's mouth. A vibration unit is integrated into the handle and the vibration unit has an output shaft extending outwardly from the handle. The output shaft vibrates when the vibration unit is turned on. A plurality of heads is provided and a respective one of the heads is removably attachable to the output shaft of the vibration unit such that the vibration vibrates the respective head.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an oral hygiene assembly according to an embodiment of the disclosure.

FIG. 2 is an exploded perspective view of an embodiment of the disclosure.

FIG. 3 is a left side perspective view of an embodiment of the disclosure.

FIG. 4 is a front view of an embodiment of the disclosure.

FIG. 5 is a cross sectional view taken along line of an embodiment of the disclosure.

FIG. 6 is a perspective view of an alternative embodiment of the disclosure.

FIG. 7 is a perspective phantom view of an alternative embodiment of the disclosure.

### DETAILED DESCRIPTION OF THE INVENTION

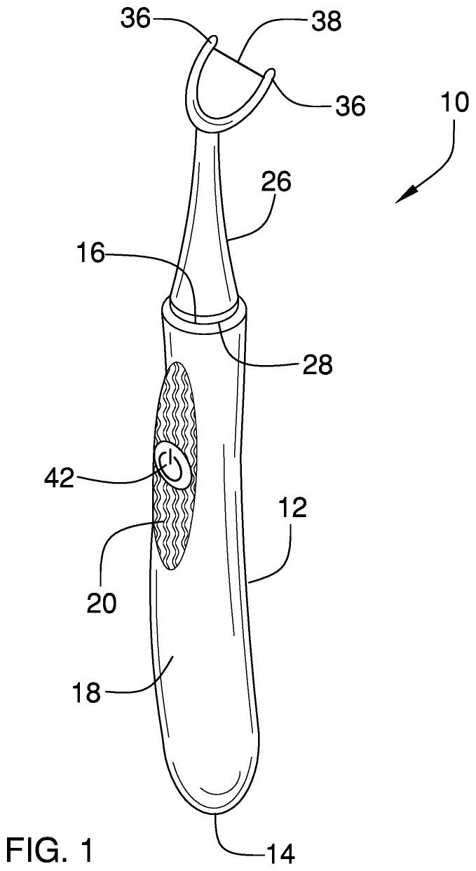

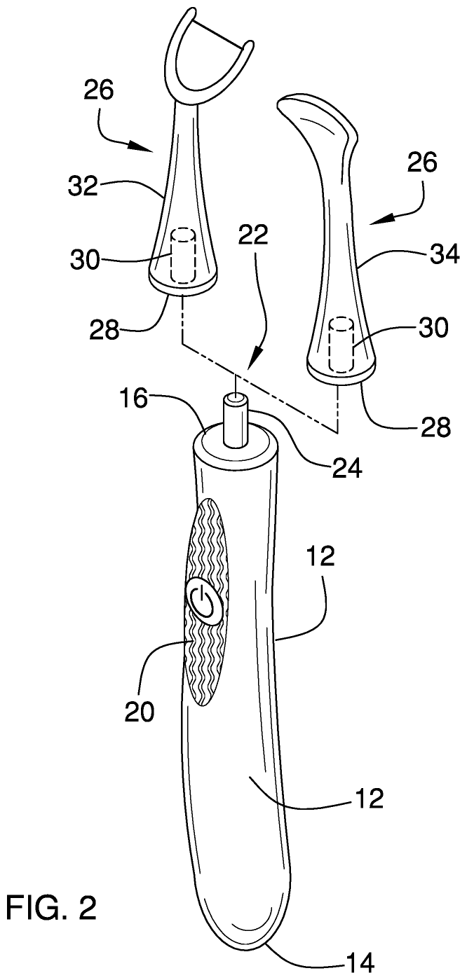

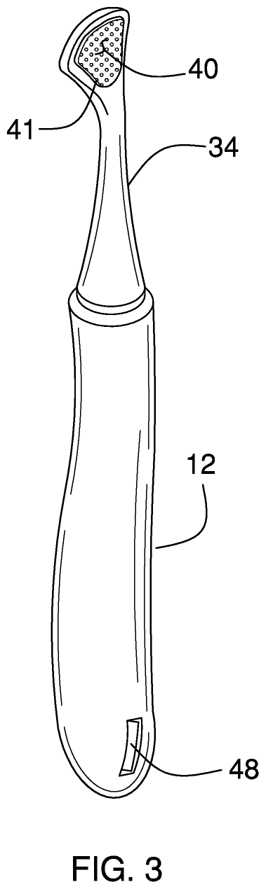

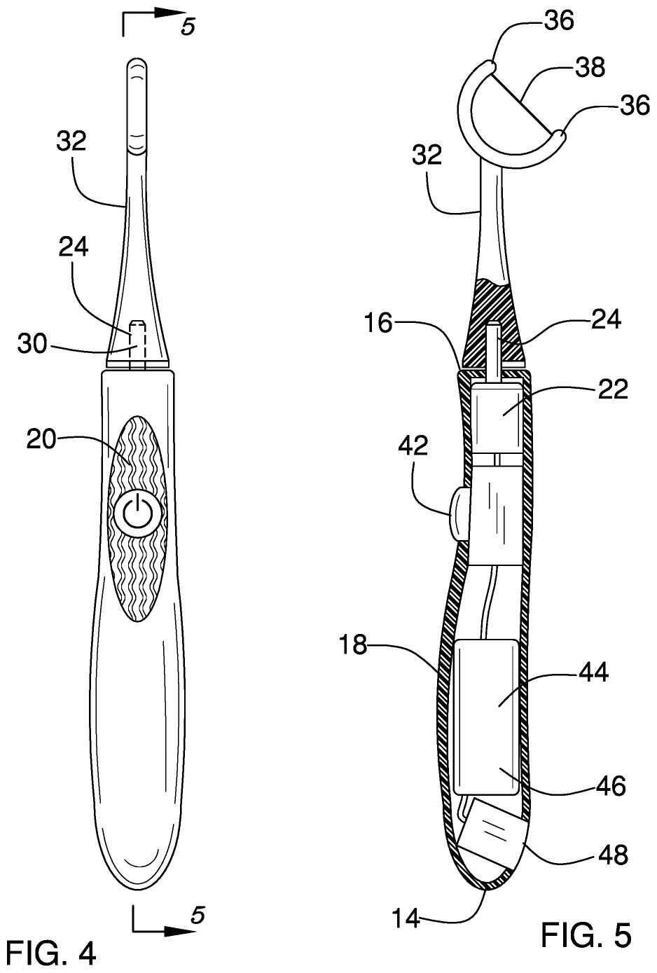

With reference now to the drawings, and in particular to FIGS. 1 through 7 thereof, a new hygiene device embodying the principles and concepts of an embodiment of the disclosure and generally designated by the reference numeral 10 will be described.

As best illustrated in FIGS. 1 through 7, the oral hygiene assembly 10 generally comprises a handle 12 that is elongated. The handle 12 has a lower end 14, an upper end 16 and an outer wall 18 extending therebetween. The lower end 14 is rounded, the upper end 16 is flattened and the handle 12 is hollow. Additionally, the outer wall 18 undulates between the lower end 14 and the upper end 16 at strategic locations to enhance a user's ability to grip the handle 12. A cushion 20 is coupled to the outer wall 18 and the cushion 20 is positioned at a strategic location to enhance comfort for the user.

A vibration unit 22 is provided and the vibration unit 22 is integrated into the handle 12. The vibration unit 22 has an output shaft 24 extending outwardly from the handle 12 and the output shaft 24 vibrates when the vibration unit 22 is turned on. Moreover, the output shaft 24 extends outwardly through the upper end 16 of the handle 12. The vibration unit 22 may comprise an electric motor that has an eccentric axis of rotation to produce vibration when the electric motor is turned on.

A plurality of heads 26 is provided and a respective one of the heads 26 is removably attachable to the output shaft 24 of the vibration unit 22 such that the vibration unit 22 vibrates the respective head 26. Each of the heads 26 has a bottom end 28, the bottom end 28 has a well 30 extending inwardly therein and the well 30 in the respective head engages the output shaft 24. The plurality of heads 26 includes a flossing head 32 and a tongue scraping head 34. The flossing head 32 includes a pair of fingers 36 that are spaced apart from each other and a line 38 extending between the fingers 36 for flossing between teeth. The fingers 36 are distally positioned with respect to the bottom end 28. The tongue scraping head 34 has a first surface 40 that is flattened and the first surface 40 is textured for scraping the user's tongue. The tongue scraping head 34 may include a scraping pad 41 that is positioned on the first surface 40 for scraping the user's tongue. Additionally, the first surface 40 is distally positioned with respect to the bottom end 38. Each of the fingers 36 is oriented to be vertically aligned with each other. Additionally, the fingers 36 extend away from the flossing head 32 at an upward angle with respect to the bottom end 28 of the flossing head 32.

A power button 42 is movably positioned on the handle 12 and the power button 42 is electrically coupled to the vibration unit 22. The power button 42 turns the vibration unit 22 on and off. A power supply 44 is integrated into the handle 12 and the power supply 44 is electrically coupled to the power button 42. The power supply 44 comprises at least one rechargeable battery 46 that is positioned within the handle 12. The at least one rechargeable battery 46 is electrically coupled to the power button 42. The power supply 44 includes a power port 48 that is recessed into the outer wall 18 of the handle 12 to receive a charge cord. The power port 48 is electrically coupled to the at least one rechargeable battery 46 for charging the at least one rechargeable battery 46.

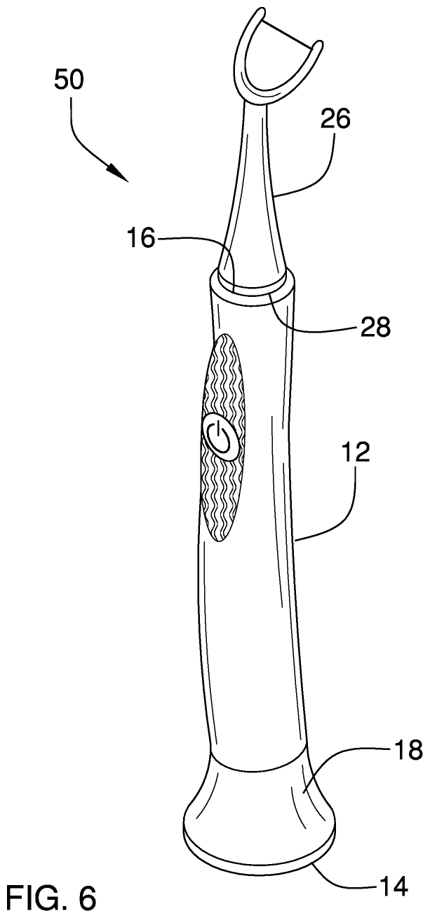

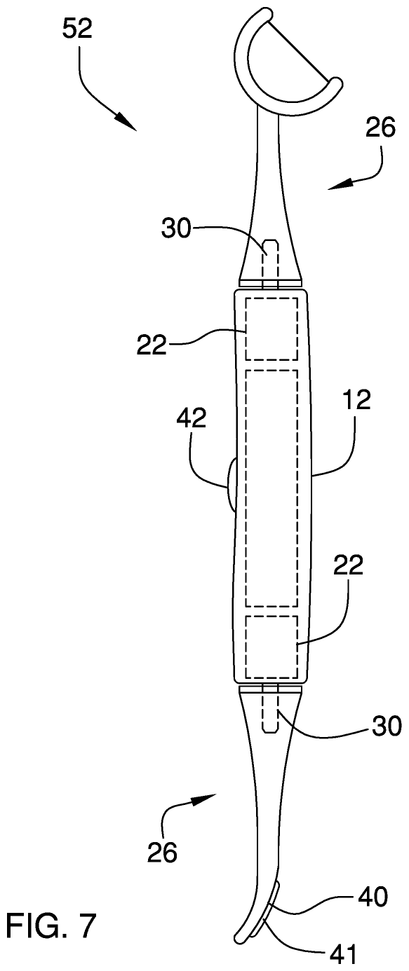

In an alternative embodiment 50 as is most clearly shown in FIG. 6, the lower end 14 of the handle 12 is flattened and the outer wall 18 flares outwardly adjacent to the lower end 14 wherein the lower end 14 is flattened. In an alternative embodiment 52 as is most clearly shown in FIG. 7, a pair of the vibration units 22 is provided. The output shaft 24 of each of the vibration units 22 extends through a respective one of the lower end 14 and the upper end 16 of the handle 12. Additionally, each of the heads 26 is attachable to the output shaft 24 of a respective one of the vibration units 22.

In use, the chosen head 26 is attached to the output shaft 24 depending on the user's preference for flossing their teeth or cleaning their tongue. The power button 42 is depressed to turn on the vibration unit 22 for vibrating the chosen head 26. In this way the process of flossing teeth or cleaning the tongue is enhanced with vibration. The rechargeable battery 46 is charged as is necessary to ensure normal operation.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An oral hygiene assembly for flossing and tongue scraping, said assembly comprising:
a handle being elongated wherein said handle is configured to reach into a user's mouth;
a vibration unit being integrated into said handle, said vibration unit having an output shaft extending outwardly from said handle, said output shaft vibrating when said vibration unit is turned on; and
a plurality of heads, a respective one of said heads being removably attachable to said output shaft of said vibration unit such that said vibration vibrates said respective head.

2. The assembly according to claim 1, wherein:
said handle has a lower end, an upper end and an outer wall extending therebetween, said lower end being rounded, said upper end being flattened, said outer wall undulating between said lower end and said upper end at strategic locations wherein said outer wall is configured to enhance a user's ability to grip said handle, said handle being hollow; and
said output shaft extends outwardly through said upper end of said handle.

3. The assembly according to claim 2, wherein each of said heads has a bottom end, said bottom end having a well extending inwardly therein, said well in said respective head engaging said output shaft.

4. The assembly according to claim 1, wherein:
said plurality of heads including a flossing head and a tongue scraping head;
said flossing head including a pair fingers being spaced apart from each other and a line extending between said fingers wherein said line is configured to floss between teeth; and
said tongue scraping head has a first surface being flattened, said first surface being textured wherein said first surface is configured to scrape the user's tongue.

5. The assembly according to claim 1, further comprising a power button being movably positioned on said handle, said power button being electrically coupled to said vibration unit, said power button turning said vibration unit on and off.

6. The assembly according to claim 5, further comprising a power supply being integrated into said handle, said power supply being electrically coupled to said power button, said power supply comprising:
at least one rechargeable battery being positioned within said handle, said at least one rechargeable battery being electrically coupled to said power button; and
a power port being recessed into said outer wall of said handle wherein said power port is configured to receive a charge cord, said power port being electrically coupled to said at least one rechargeable battery for charging said at least one rechargeable battery.

7. An oral hygiene assembly for flossing and tongue scraping, said assembly comprising:
a handle being elongated wherein said handle is configured to reach into a user's mouth, said handle having a lower end, an upper end and an outer wall extending therebetween, said lower end being rounded, said upper end being flattened, said outer wall undulating between said lower end and said upper end at strategic locations wherein said outer wall is configured to enhance a user's ability to grip said handle, said handle being hollow;
a vibration unit being integrated into said handle, said vibration unit having an output shaft extending outwardly from said handle, said output shaft vibrating when said vibration unit is turned on, said output shaft extending outwardly through said upper end of said handle;
a plurality of heads, a respective one of said heads being removably attachable to said output shaft of said vibration unit such that said vibration vibrates said respective head, each of said heads having a bottom end, said bottom end having a well extending inwardly therein, said well in said respective head engaging said output shaft, said plurality of heads including a flossing head and a tongue scraping head, said flossing head including a pair fingers being spaced apart from each other and a line extending between said fingers wherein said line is configured to floss between teeth, said tongue scraping head having a first surface being flattened, said first surface being textured wherein said first surface is configured to scrape the user's tongue;
a power button being movably positioned on said handle, said power button being electrically coupled to said vibration unit, said power button turning said vibration unit on and off; and
a power supply being integrated into said handle, said power supply being electrically coupled to said power button, said power supply comprising:
at least one rechargeable battery being positioned within said handle, said at least one rechargeable battery being electrically coupled to said power button; and
a power port being recessed into said outer wall of said handle wherein said power port is configured to receive a charge cord, said power port being electrically coupled to said at least one rechargeable battery for charging said at least one rechargeable battery.

8. The assembly according to claim 7, wherein said lower end of said handle is flattened, said outer wall flaring outwardly adjacent to said lower end wherein said lower end is configured to rest on a support surface.

9. The assembly according to claim 7, wherein said lower end of said handle is flattened.

10. The assembly according to claim 9, wherein a pair of said vibration units is provided, said output shaft of each of said vibration units extending through a respective one of said lower end and said upper end of said handle.

11. The assembly according to claim 10, wherein each of said heads is attachable to said output shaft of a respective one of said vibration units.
